(19) United States
(12) Patent Application Publication
(10) Pub. No.: US 2015/0342795 A1
(43) Pub. Date: Dec. 3, 2015

(54) ABSORBENT ARTICLE WITH MULTIPLE LAYERS

(71) Applicant: PRODUCTOS FAMILIA S.A., Medellin (CO)

(72) Inventors: Andrea Alzate Machado, Medellin (CO); Fabio Alberto López Pimienta, Medellin (CO); Juan Manuel Chamat, Medellin (CO)

(21) Appl. No.: 14/660,169

(22) Filed: Mar. 17, 2015

(30) Foreign Application Priority Data
May 27, 2014 (CO) 14-114.349

(51) Int. Cl. A61F 13/511; A61F 13/56; A61F 13/472; A61F 13/514; A61F 13/47

(52) U.S. Cl. CPC A61F 13/5116; A61F 13/51478; A61F 13/4704; A61F 13/47245; A61F 13/5616; A61F 2013/5683

(57) ABSTRACT

The present invention corresponds to an absorbent article comprising substrates having a top layer and a bottom layer, wherein the substrates are located one on top of the other. The bottom substrate having attachment media located on the bottom layer, and the top substrate having support media located on the top end and bottom end of the top substrate. The bottom and op substrates are attached throughout the periphery thereof, and the top layer is permeable.

### FIELD OF THE INVENTION

The invention refers to an absorbent article comprising several substrates, with or without wings, located one on top of the other comprising one top permeable layer and one bottom impermeable layer bound together throughout the periphery. The absorbent article also comprises an absorbent core having different geometrical shapes.

### DESCRIPTION OF THE ART

Amongst the wide range of personal hygiene products available in the marketplace, there are the commonly used body fluid capture or retention products. Said absorbent articles, such as sanitary towels, daily protectors and incontinency absorbers are widely known and differ essentially in their shape, size, manufacturing process and absorption capacity.

Absorbent articles for female personal hygiene must be changed several times during the day, implying the user must discard the one in use and replace it with a new one. This in some occasions may generate inconvenience for the user, given a sufficiently private place is not always readily available for changing and in addition, several of these articles must be carried.

Several ways to solve this inconvenience have been disclosed. CO 08088369 describes a protector-type absorbent article having several removable layers with interdetachment media, in a way that every time the protector needs changing for a new one, the top layer is detached. However, said interdetachment medium causes the absorbent article to only have one position during use and thus, one sole possible place to detach the top layer. The proposed solution in the cited document is only described for daily protectors and does not address other absorbent articles such as sanitary towels.

In addition, female hygiene products disclosed in the art in most cases have been designed to fit a specific size of underwear reinforcement and not to the different shapes thereof, and thus when acquiring a product for normal underwear, it is not possible to be used for thong-type underwear or vice versa.

### BRIEF SUMMARY

The present invention refers to an absorbent article comprising substrates located one on top of the other comprising a top permeable layer and a bottom impermeable layer, and may also comprise an absorbent core, in which case the top layer and bottom layer are folded with respect to the edges of the absorbent core which has an ovoid, oval, rounded triangle, or other shapes. The bottom substrate contains attachment media located on the bottom layer, whilst the top substrate has support media located towards its top end and bottom end. The bottom and top substrates are held together throughout the periphery. The substrates comprising the absorbent article may or may not have wings. The invention may comprise substrates having a distributing layer located between the top layer and the absorbent core. The bottom layer of the top substrates may be liquid permeable.

### BRIEF DESCRIPTION OF THE DRAWINGS

FIG. 1. Illustrates the different substrates which comprise the absorbent article.

FIG. 2. Illustrates the attachment media of a substrate

FIG. 3. Embodiment of the invention wherein the absorbent article is comprised of substrates having wings

FIG. 4. Embodiment of the invention wherein the absorbent article is comprised of substrates having wings and substrates without wings

FIG. 5. Embodiment of the invention wherein the absorbent article is comprised of substrates without wings

FIG. 6. Top substrate. Illustrates side borders, bottom end, top end and longitudinal axis of a substrate.

FIG. 7. Embodiment of the invention comprising two support media located over the longitudinal axis, one at the bottom end and the other at the top end of the substrate.

FIG. 8. Illustrates the folding lines with respect to the periphery of the absorbent core which are described when folding the top layer and the bottom layer.

### DETAILED DESCRIPTION OF THE INVENTION

The present invention relates to an absorbent article comprised of at least two adjacent substrates, preferably one on top of the other, one of them being the top substrate (4) and the other the bottom substrate (5). If the article is comprised of three or more substrates, the middle substrates are located between the top substrate (4) and bottom substrate (5). Each substrate has several support means (9), giving the user flexibility in order to place the absorbent article in two positions. In addition, the absorbent article described herein is adaptable to several types of underwear, thus providing the user more flexibility for use with different types of underwear.

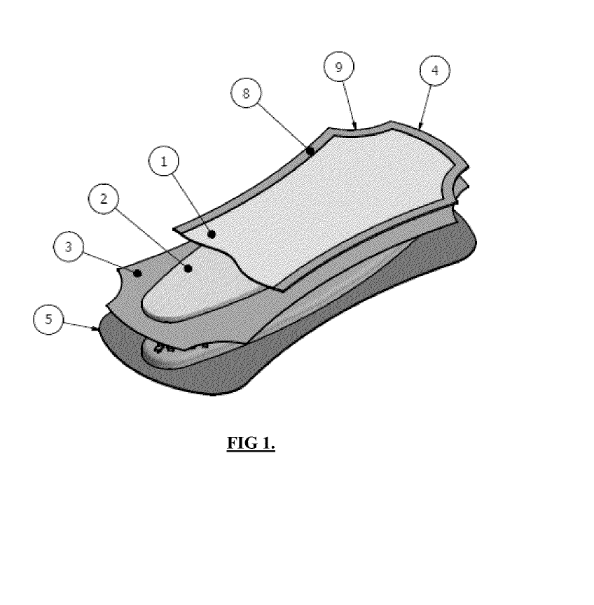

According to FIG. 1, in one embodiment of the invention, the substrates of the absorbent article are located one on top of the other in such a manner that the top layer (1) of the top substrate (4) contacts the user. The bottom layer (3) of the top substrate (4) contacts the top layer (1) of the bottom substrate (5). Each substrate is comprised of a liquid permeable top layer (1), an absorbent core (2) and a bottom layer (3).

The bottom layer (3) is liquid-impermeable and contacts the underwear or the top layer (1) of the bottom substrate (5). The bottom layer (3) avoids that fluids absorbed by the top substrate (4) reach the underwear or come into contact with the bottom substrate (5). Absorbent core (2) is found between the top layer (1) and the bottom layer (3) which is comprised of one or more layers, of one or more materials such as pulp, tissue, SAP and blends thereof, in order to absorb and retain captured fluids.

In an embodiment of the invention, the bottom layer (3) present in the top substrates (4) may be liquid permeable in order to provide greater absorption capacity to the absorbent core.

In an embodiment of the invention, the substrate does not have an absorbent core (2) and only comprises a top layer (1) and a bottom layer (3), wherein the top layer (1) absorbs and retains fluids.

In an embodiment of the invention (not illustrated), the top substrates (4) randomly have an absorbent core (2) between the top layer (1) and the bottom layer (3).

In an embodiment of the invention (not illustrated), a distributing layer is located between the top layer (1) and the absorbent core (2) which is in charge of distributing fluids that are absorbed by the absorbent core (2).

According to FIG. 1, top substrate (4) and bottom substrate (5) which comprise the absorbent article are located on the periphery (8) in order to avoid fluids leaks from the top substrate (4) towards the bottom substrate (5). The attachment allows to separate the substrates without compromising their integrity. The attachment is possible by mechanical weaving, adhesives, hotmelts, ultrasound or a combination thereof.

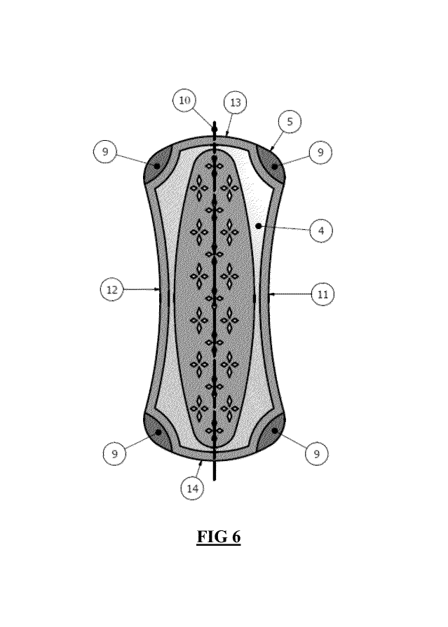

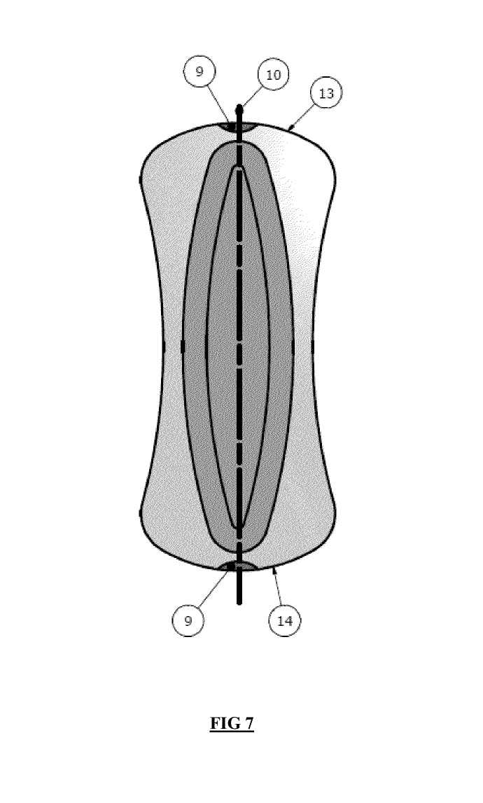

According to FIG. 6, substrates (4) and (5) describe (FIG. 6) an elongated surface having a longitudinal axis (10). On the substrate, four ends are identified, two side ends (11) and (12) located on either side of longitudinal axis (10), and another two ends intersected by the longitudinal axis (10) which correspond to top end (13) and bottom end (14).

According to FIG. 1, substrate (4) comprises support means (9). Support means (9) can be folds, curled folds, precuts, or strips located on the top layer (1) or bottom layer (3) of the top substrate (4). Through the use of the support means (9), the user may easily detach a substrate from the absorbent article by pulling on the substrate intended for detachment. According to FIG. 6, the support means (9) are located on the periphery (8) of top end (13) and of bottom end (14) of the top substrate (4).

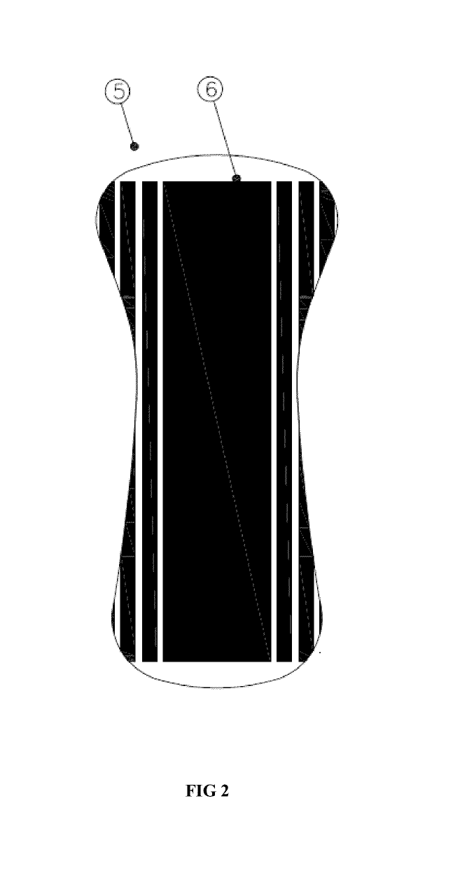

In an additional embodiment of the invention, according to FIG. 2, bottom substrate (5) comprises attachment means (6) in order to attach the absorbent article to the underwear and thus avoids the absorbent article from moving. The attachment means (6) may be adhesives, Velcro, amongst others, and adheres to the bottom layer (3) through the face opposite to the one where the absorbent core (2) is found.

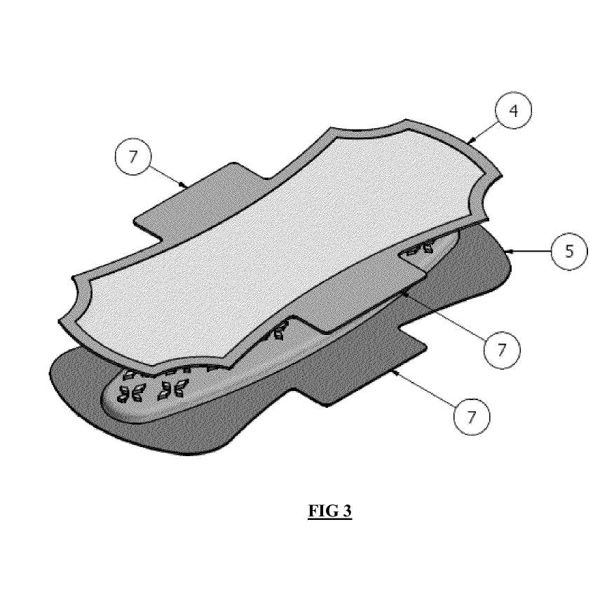

In an embodiment of the invention, according to FIG. 3, substrates (4) and (5) which comprise the absorbent article, have wings (7). The wings (7) of the top substrates (4) may be the same, bigger or smaller in size than the wings (7) of the bottom substrate (5).

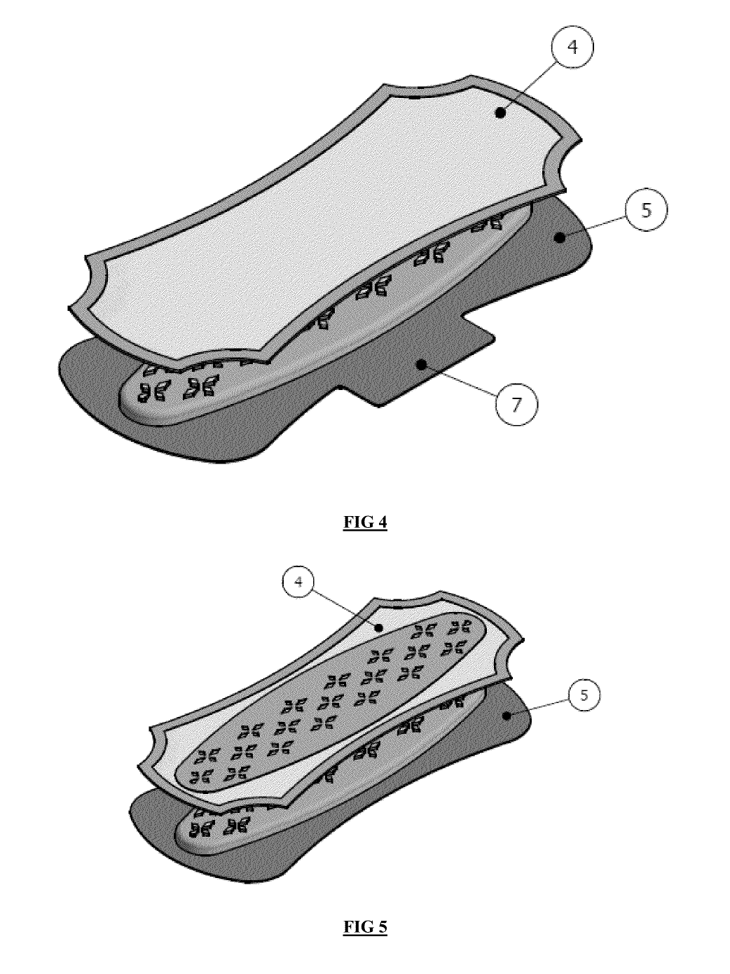

In an embodiment of the invention, according to FIG. 4, the bottom substrate (5) has wings (7) and the top substrates (4) do not have wings (7).

In an embodiment of the invention, according to FIG. 5, the bottom substrate (4) and the top substrate (5) comprising the absorbent article, do not have wings (7).

In an embodiment of the invention (not illustrated), the substrates comprising the absorbent article, alternate in having and not having wings (7).

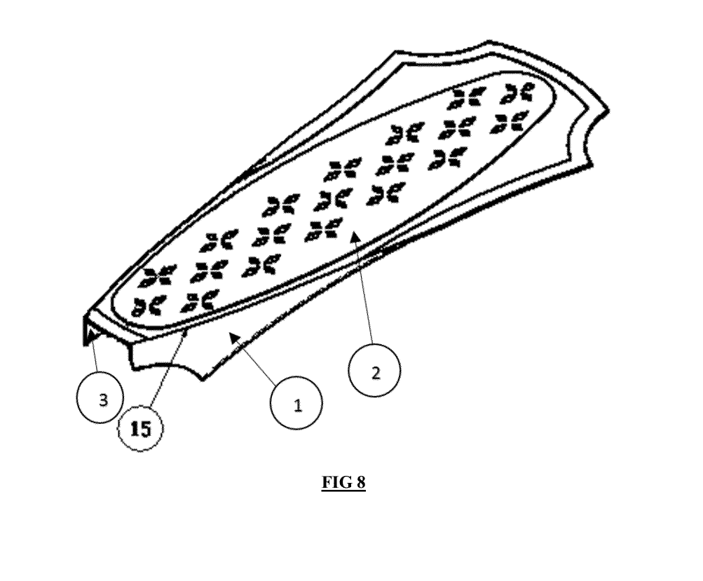

In an embodiment of the invention, according to FIG. 7, each substrate has at least two support means (9), one on the top end (13) and another on the bottom end (14) over the longitudinal axis (10). In another embodiment of the invention (not illustrated), the support means are located one on the top end (13) and the other on the bottom end (14), not necessarily over the longitudinal axis (10). In an additional embodiment, according to FIG. 6, the substrates have four support means (9), two on the top end (13) and two on the bottom end (14).

When support means (9) are present on the substrates, both on the top end (13) and bottom end (14), the position for use of the absorbent article is indifferent.

According to FIG. 1, the absorbent core (2) of the top substrate (4) and of the bottom substrate (5) comprising the absorbent article, has an ovoid shape, although it could be oval shaped, rounded triangle shaped, or similarly shaped. In addition, the shape of the absorbent core (2) has a greater thickness than the top layer (1) and bottom layer (3); thus, and according to FIG. 1, the absorbent core (2) describes a volume which can have one of the aforementioned shapes. According to FIG. 8, the shape of the absorbent core (2) allows for the top layer (1) and bottom layer (3) to fold describing the folding lines (15) with respect to the periphery of the absorbent core (2) or underwear; the folding lines (15) are tangential to the periphery of the absorbent core (2).

In an embodiment of the invention (not illustrated), the absorbent core (2) of one or several of the substrates comprising the absorbent article, two portions are observed: a first portion corresponding to a shape located inside the absorbent core (2) and a second portion corresponding to a ring enclosing the first portion. The shape of the first portion of the absorbent core has an ovoid, oval, rounded triangle or similar shape. The shape in the absorbent core allows for the top layer (1), the second portion of the absorbent core (2) and the bottom layer (3) to fold with respect to the periphery of the shape of the first portion of the absorbent core (2), thus describing a folding line (15) with respect to the periphery of the shape of the first portion of the absorbent core (2) or the underwear. Upon projecting said folding lines (15), these do not intersect inside the absorbent article implying that each folding line (15) forms an angle of less than 90° with respect to longitudinal axis (10), and the point where the folding lines (15) intersect will never fall inside the absorbent article.

In an embodiment of the invention (not illustrated), the shape of the absorbent core (2) is asymmetrical with respect to the cross-section axis. Said cross-section axis is perpendicular to the longitudinal axis (10), and therefore, the shape of the absorbent core (2) is an indicator of the position of use.

It must be understood that the present invention is limited to the embodiments described and illustrated herein, and as will be evident to any normally skilled person in the art, possible variations and modifications exist that do not fall outside the scope of the invention, which is solely defined by the following claims.

1. An absorbent article having substrates located one on top of the other, each substrate including a top permeable layer and a bottom layer, where the absorbent article comprises:
a top substrate having support means located on a top end and a bottom end;
a bottom substrate having attachment means located on the bottom layer;
wherein the bottom and top substrates are attached throughout the periphery thereof, and the bottom layer of the bottom substrate is impermeable.

2. The absorbent article claimed in claim 1, comprising more than one top substrate.

3. The absorbent article claimed in claim 1, characterized because the bottom substrate has an absorbent core between the top permeable layer and the bottom impermeable layer.

4. The absorbent article claimed claim 1, characterized because the substrates have an absorbent core between the top permeable layer and the bottom layer.

5. The absorbent article claimed in claim 2, characterized because the top substrates randomly have an absorbent core between the top permeable layer and bottom layer.

6. The absorbent article claimed in claim 1, characterized because the substrates have wings.

7. The absorbent article claimed in claim 6, characterized because the size of the wings of the top substrate, with respect to the wings of the bottom substrate, is selected from the group consisting of the same size, smaller size, or bigger size.

8. (canceled)

9. (canceled)

10. The absorbent article claimed in claim 1, characterized because only the bottom substrate has wings.

11. The absorbent article claimed in claim 1, characterized because the top substrate comprises one support means on the top end and one support means on the bottom end.

12. The absorbent article claimed in claim 1, characterized because the top substrate comprises two support means on the top end and two support means on the bottom end.

13. The absorbent article claimed in claim 1, characterized because the support means are located on the top permeable layer of the top substrate.

14. The absorbent article claimed in claim 1, characterized because the support means are located on the bottom layer of the top substrate.

15. The absorbent article claimed in claim 3, characterized because the shape of the absorbent core of the substrates is ovoid, oval or, rounded-triangle.

16. The absorbent article claimed in claim 15, characterized because the top permeable layers and the bottom layers of the substrates fold with respect to the periphery of the absorbent core or underwear, describing folding lines which are tangential to the periphery of the absorbent core.

17. The absorbent article claimed in claims 3, characterized because the absorbent core contains:
a first portion of ovoid, oval, rounded-triangle or a combination thereof, located inside the absorbent core; and,
a second portion corresponding to a ring enclosing the first portion.

18. The absorbent article claimed in claim 17, characterized because the top permeable layers, bottom layers and the second portion of the absorbent core of the substrates fold with respect to the periphery of the shape of the first portion of the absorbent core, describing folding lines at an angle of less than or equal to 90° with respect to the longitudinal axis of the substrates and an intersecting point of the folding lines is located outside the absorbent article.

19. The absorbent article claimed in claims 3, characterized because the shape of the substrates is asymmetrical to a cross-section axis.

20. The absorbent article claimed in claims 3, characterized because a substrate comprises a distributing layer between the top permeable layer and the absorbent core.

21. The absorbent article claimed in claim 1, characterized because the bottom layer of the top substrates is permeable.

22. The absorbent article of claimed in claim 1, characterized because the bottom layer of the top substrates is impermeable.

23. (canceled)
